(19) United States
(12) Patent Application Publication
(10) Pub. No.: US 2021/0015675 A1
(43) Pub. Date: Jan. 21, 2021

(54) EAR COVERING PROTECTOR

(71) Applicant: Pearline Sturridge, Etobicoke (CA)

(72) Inventor: Pearline Sturridge, Etobicoke (CA)

(21) Appl. No.: 16/517,633

(22) Filed: Jul. 21, 2019

(51) Int. Cl. A61F 11/12; A61F 11/14; A45D 44/12

(52) U.S. Cl. CPC A61F 11/12; A45D 44/12; A61F 11/14

(57) ABSTRACT

An illustrated view of an exemplary ear protection device for protecting an outer ear. The ear protection device is useful for protecting the outer ear from being subject to cuts or nicks from scissors or other sharp articles, unwanted dye or other hair solution on ear skin, burns from styling equipment, hair clippings or other items during the cutting of the hair affecting the ear skin, etc. Additionally, the ear protection device helps alleviate bug, such as mosquitos, flies, etc., from entering the ear while sitting at a camp site, front yard, back yard, etc. The ear protection device has an ear-plug and a cover and an ear-plug integrated to an inner face of the cover. A logo may be on an outer face of the cover.

### FIELD OF THE INVENTION

This invention relates to ear coverings. More particularly, it relates to a protective ear covering device.

### BACKGROUND

The ear is the organ of hearing and, in mammals, balance. In mammals, the ear is usually described as having three parts—the outer ear, the middle ear and the inner ear. The outer ear consists of the pinna and the ear canal. Since the outer ear is the only visible portion of the ear in most animals, the word “ear” often refers to the external part alone. The middle ear includes the tympanic cavity and the three ossicles. The inner ear sits in the bony labyrinth, and contains structures which are key to several senses: the semicircular canals, which enable balance and eye tracking when moving; the utricle and saccule, which enable balance when stationary; and the cochlea, which enables hearing. The ears of vertebrates are placed somewhat symmetrically on either side of the head, an arrangement that aids sound localization.

An earplug is a device that is inserted in the ear canal to protect the user's ears from loud noises, intrusion of water, foreign bodies, dust or excessive wind. Since they reduce the sound volume, earplugs are often used to help prevent hearing loss and tinnitus (ringing of the ears).

There are mainly four types of earplug fittings for hearing protection 1) Foam earplugs, mainly made from either polyvinyl chloride (PVC) or polyurethane (PU) (memory foam), which are compressed (rolled) and put into the ear canal, where they expand to plug it; 2) Wax earplugs, which are rolled into a ball and carefully molded to fit over the external portion of the ear canal; 3) Flanged silicone earplugs, an example of Universal-fit; 4) Custom molded earplugs, made from a mold of the wearer's ear and designed to precisely fit all ear canal shapes. Custom molded is further divided into laboratory-made and “formed in place”.

NIOSH Mining Safety and Health Research recommends using the roll, pull, and hold method when using memory foam earplugs. The process involves the user rolling the earplug into a thin rod, pulling back on the ear, and holding the earplug deep in the canal with the finger. To get a complete seal, the user must wait about 20 to 30 seconds for the earplug to expand inside the canal.

Earplugs are most effective when the wearer has been properly trained on use and insertion. Employers can provide this training before dispensing earplugs to their employees. Training for earplug use includes: insertion, a seal check, depth check, removal, cleaning, and replacement. When training on insertion, the objective is for the worker to understand the correct insertion strategy.

Proper insertion training prevents inadequate insertion, that can result in discomfort or inadequate attenuation, which can result in hearing loss. When this step is achieved, then the seal and depth need to be checked. The ear-plugs all have a desired depth and seal which need to be achieved to provide the designated attenuation for the wearer. The worker will also be trained on how to properly remove the earplugs and dean them. This allows for multiple uses and reduces the chance of infection. To further prevent infection, it is important the worker understands when they will need to replace their earplugs. Once the plugs have been worn down from repeated use, they will no longer seal correctly or provide the proper attenuation level, and the device will need to be replaced.

Earplugs and other hearing protection devices can be tested to ensure that they fit properly and are successfully limiting sound exposure with a number of different systems, most of which use large noise-cancelling headphones that fit over the ear and transmit the test sounds.

Although most earplugs protect or are effective for the ear canal, the outer ear can be subject cuts or nicks from scissors or other sharp articles, unwanted dye or other hair solution on ear skin, burns from styling equipment, hair clippings or other items during the cutting of the hair affecting the ear skin, etc.

In light of the foregoing, there is a need for a device to protect the outer portion of the ear or ear skin from these issues.

### BRIEF DESCRIPTION OF THE DRAWINGS

FIG. 1 is an illustrated view of an exemplary ear protection device.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

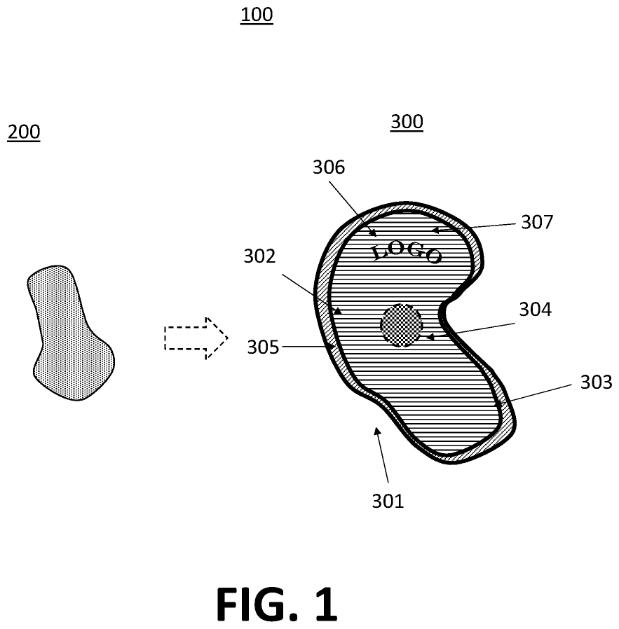

Referring to FIG. 1, an illustrated view of an exemplary ear protection device 100 for protecting an outer ear. The ear protection device 100 is useful for protecting the outer ear from being subject to cuts or nicks from scissors or other sharp articles, unwanted dye or other hair solution on ear skin, burns from styling equipment, hair clippings or other items during the cutting of the hair affecting the ear skin, etc. Additionally, the ear protection device 100 helps alleviate bug, such as mosquitos, flies, etc., from entering the ear while sitting at a camp site, front yard, back yard, etc.

The ear protection device 100 has an ear-plug 200 and a cover 300. The ear-plug 200 is useful for being coupled to an ear canal to protect the ear canal from injury, protect items from entering the ear, to reduce noise being heard or to help ease various ailments such as ringing of the ears. The ear-plug 200 is preferably made of a foam material, but other materials are hereby contemplated, including, but not limited to, rubber, plastic, etc.

The cover 300 has an inner face 301, an outer face 302, an outer edge 303, a center 304 and a lip 305. The cover 300 is preferably a size of an ear. The cover 300 is preferably made of a foam material, but other materials are hereby contemplated, including, but not limited to, rubber, plastic, cloth, etc. The material is preferably flexible.

The cover 300 is placed over the outer ear and coupled to the outer edge of the outer ear. The lip 305 of the outer edge 303 of the cover 300 is coupled to an outer edge of the outer ear.

The ear-plug 200 is coupled to the inner face 301 of the cover 300, thus it is integrated into the ear cover 300.

Optionally and/or additionally, a logo 306 may be embossed onto an upper portion 307 of the outer face 302 of the cover 300.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ear protective device for protecting an ear, the device comprising:
a cover, the cover comprising:
an inner face;
an outer face, the outer face having a lip, wherein the lip being for coupling to the outer edge of the outer ear; and
an ear-plug, the ear-plug being integral to the inner surface of the cover, wherein the ear-plug for coupling to an ear canal.

2. The device of claim 1, wherein the cover being made of a foam material.

3. The device of claim 2, wherein the material being flexible.

4. The device of claim 1, wherein the ear-plug being made of a foam material.

5. The device of claim 1, wherein the device being a same size of an ear.

6. The device of claim 1, wherein the outer face having a logo on a center portion of the outer face.
